(19) United States
(12) Patent Application Publication
(10) Pub. No.: US 2020/0008465 A1
(43) Pub. Date: Jan. 9, 2020

(54) ATOMIZER AND ASPIRATION DEVICE WITH ATOMIZATION FUNCTION

(71) Applicant: SHENZHEN UWELL TECHNOLOGY CO., LTD., Shenzhen (CN)

(72) Inventor: YUCHENG XIAO, Shenzhen (CN)

(21) Appl. No.: 16/039,296

(22) Filed: Jul. 18, 2018

(30) Foreign Application Priority Data
Jul. 9, 2018 (CN) 201810745898.3

(51) Int. Cl. A24F 47/00; A61M 11/04

(52) U.S. Cl. CPC A24F 47/008; A61M 2205/3331; A61M 11/042

(57) ABSTRACT

An atomizer includes a central region, a marginal region, a liquid storage tank assembly defining a first hole, a base assembly including a base body including an accommodating room and inlet holes, and an atomization core assembly. The atomization core assembly includes an atomizing member including an atomizing room and a diverting member including a diverting room including a swirling flow region having a second hole and a straight flow region including first airflow holes and first diverting plates. Air flows into the accommodating room from the inlet holes, one part of the air flows through the second hole, the atomizing room, and the first hole in a swirling state, and forms a swirling flow in the central region; the other part of the air flows through the first airflow holes, the atomizing room, the first hole in a straight state, and forms a straight flow in the marginal region.

### 1. TECHNICAL FIELD

The present invention relates to substitutes of cigarette, in particular to relates to an atomizer and an aspiration device with atomization function.

### 2. DESCRIPTION OF RELATED ART

The present atomizer on the market only has a swirling flow member or a straight flow member, so that the atomizer cannot keep the stability of the smoke's taste while improve the heat dissipation performance.

### SUMMARY

In order to overcome the above problems, the purpose of the present invention is to provide an atomizer that can keep the stability of the smoke's taste and improve the heat dissipation performance.

The technical solution adopted for solving technical problems of the present disclosure is:

An atomizer includes a central region, a marginal region, a liquid storage tank assembly defining a first hole, a base assembly including a base body, and an atomization core assembly located between the liquid storage tank assembly and the base assembly. The base body includes an accommodating room and a plurality of inlet holes in communication with the accommodating room. The atomization core assembly includes an atomizing member and a diverting member assembled in the atomizing member. The atomizing member includes an atomizing room, and the diverting member includes a diverting room including a swirling flow region and a straight flow region in communication with the swirling flow region. The swirling flow region defines a second hole, the straight flow region includes a plurality of first airflow holes and a plurality of first diverting plates, the second hole and the plurality of first airflow holes in communication with the atomizing room. Wherein air flows into the accommodating room from the plurality of inlet holes, one part of the air flows through the second hole, the atomizing room, and the first hole in a swirling state, and forms a swirling flow in the central region; the other part of the air flows through the plurality of first airflow holes, the atomizing room, the first hole in a straight state, and forms a straight flow in the marginal region.

wherein each of the first diverting plates is arranged on an inner wall of the diverting room along a radial direction and projects from the inner wall of the diverting room to the second hole, and each of the first diverting plates is located between two adjacent first airflow holes, to facilitate the other part of the air flows through the marginal region in the straight state.

wherein cross-sections of the plurality of first diverting plates form a circle, a central axis of the circle is the same as that of the second hole.

wherein a cross-section of each of the plurality of first airflow holes is a general U shape having a slowly decreasing opening, a central axis of each of the plurality of first airflow holes is parallel to that of the second hole.

wherein the diverting member further comprises an eliminating noise portion located at a distal end thereof and a border between the swirling flow region and the straight flow region.

wherein the base body comprises a swirling flow forming member to change the air from the plurality of inlet holes into the swirling flow to flow through the central region in the swirling state.

wherein each of the plurality of first diverting plates is provided to change the swirling flow formed by the swirling flow forming member to the straight flow to flow through the marginal region in the straight state, and a cross-section of each of the plurality of first diverting plates slowly expands from the base body to the atomizing member to facilitate the swirling flow to slowly change to the straight flow.

wherein the swirling flow forming member comprises a plurality of arcuate guide vanes arranged on the inner wall of the accommodating room and extending meanderingly from the inner wall of the accommodating room to a center of the accommodating room.

wherein the liquid storage tank assembly comprises a cover, and the first hole is located in a middle portion of the cover, and wherein the cover comprises a stabilizing flow plate, and the stabilizing flow plate is arranged on an inner wall of the first hole and projecting from the inner wall of the first hole to a center of the first hole.

wherein the atomizer further comprises a deposition chamber located between the cover and the atomization core assembly to receive a plurality of small carbon particles.

The present disclosure provides the advantages as below.

The structure of the present disclosure is provided for including the straight flow in the marginal region to keeps stability of the smoke's taste and the swirling flow in the central region to improve heat dissipation performance. Simultaneously, by taking use of the swirling flow scouring condensation E-liquid in the accommodating room to the fine drops, the fine drops and the swirling flow mix into E-liquid-air mixture flowing through the atomizing member to atomize again, thereby keeping dry and clean of the accommodating room. Further, the fine drops is swung to inner walls of atomizing room and the first hole under the function of centrifugal force of the swirling flow, and the fine drops and the swirling flow are separated, thereby keeping the cleanliness of the smoke and improving the user's taste experience.

### BRIEF DESCRIPTION OF THE DRAWINGS

Many aspects of the embodiments can be better understood with reference to the following drawings. The components in the drawings are not necessarily dawns to scale, the emphasis instead being placed upon clearly illustrating the principles of the embodiments. Moreover, in the drawings, like reference numerals designate corresponding parts throughout the several views.

FIG. 1 is an assembled view of an aspiration device with atomization function of an exemplary embodiment of the present invention, the atomizer and an aspiration device with atomization function including an atomizer and a battery assembly;

FIG. 2 is a cross-sectional view of the atomizer of FIG. 1 in accordance with a first exemplary embodiment of the present disclosure;

FIG. 3 is a cross-sectional view of a base assembly of FIG. 2;

FIG. 4 is a cross-sectional view of an atomization core assembly of FIG. 2;

FIG. 5 is a cross-sectional view of a liquid storage tank assembly of FIG. 2;

FIG. 6 is a schematic view of a diverting member in accordance with a first exemplary embodiment of the present disclosure;

FIG. 7 is a top plan view of FIG. 6;

FIG. 8 is a schematic view of a diverting member in accordance with a second exemplary embodiment of the present disclosure;

FIG. 9 is a cross-sectional view of FIG. 8;

FIG. 10 is a schematic view of a base body in accordance with a first exemplary embodiment of the present disclosure;

FIG. 11 is a cross-sectional view of FIG. 10;

FIG. 12 is a schematic view of a base body in accordance with a second exemplary embodiment of the present disclosure;

FIG. 13 is a cross-sectional view of FIG. 12;

FIG. 14 is a cross-sectional view of the atomizer of FIG. 1 in accordance with a second exemplary embodiment of the present disclosure; and

FIG. 15 is a schematic view of a cover of FIG. 14 in accordance with a second exemplary embodiment of the present disclosure.

### DETAILED DESCRIPTION

The disclosure is illustrated by way of example and not by way of limitation in the figures of the accompanying drawings, in which like reference numerals indicate similar elements.

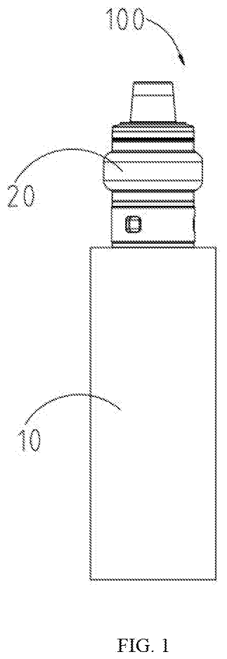

Referring to FIG. 1, the aspiration device with atomization function 100 in accordance with an exemplary embodiment of the present disclosure includes a battery assembly 10 and an atomizer 20. The battery assembly 10 is electrically connected with the atomizer 20 and supplies power for the atomizer 20.

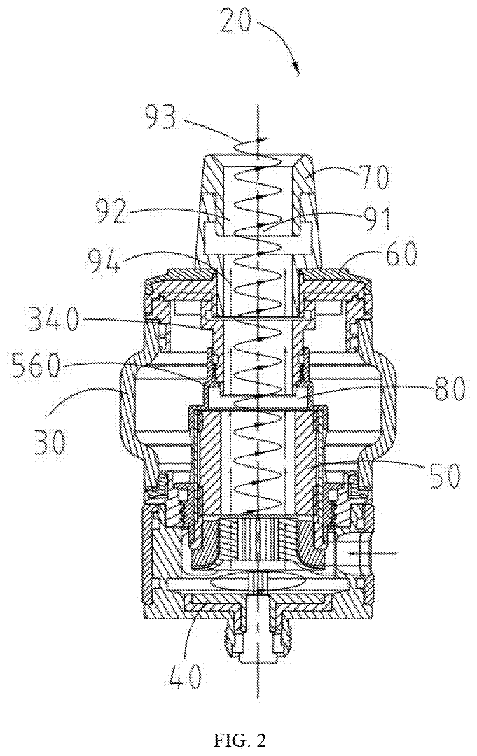

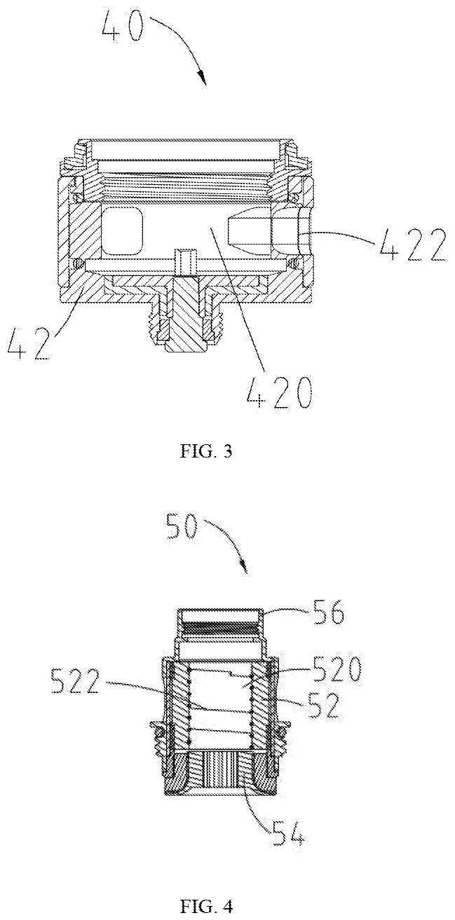

Referring to FIG. 2 to FIG. 7, the atomizer 20 includes a central region 91, a marginal region 92, a liquid storage tank assembly 30, a base assembly 40, and an atomization core assembly 50. The liquid storage tank assembly 30 defines a first hole 32. The base assembly includes a base body 42, and the base body 42 includes an accommodating room 420 and a plurality of inlet holes 422 in communication with the accommodating room 420. The atomization core assembly 50 is located between the liquid storage tank assembly 30 and the base assembly 40. The atomization core assembly 50 includes an atomizing member 52 and a diverting member 54 assembled in the atomizing member 52. The atomizing member 52 includes an atomizing room 520. The diverting member 54 includes a diverting room 540 including a swirling flow region 541 and a straight flow region 543 in communication with the swirling flow region 541. The swirling flow region 541 defines a second hole 5410. The straight flow region 543 includes a plurality of first airflow holes 5431 and a plurality of first diverting plates 5433, and the second hole 5410 and the plurality of first airflow holes 5431 are in communication with the atomizing room 520. Wherein, air flows into the accommodating room 420 from the plurality of inlet holes 422, one part of the air flows through the second hole 5410, the atomizing room 520, and the first hole 32 in a swirling state, and forms a swirling flow 93 in the central region 91; the other part of the air flows through the plurality of first airflow holes 5431, the atomizing room 520, the first hole 32 in a straight state, and forms a straight flow 94 in the marginal region 92.

In one embodiment, the swirling flow 93 flows through central regions of the atomizing room 520 and the first hole 32. The straight flow 94 flows through peripheral regions of the atomizing room 520 and the first hole 32.

In one embodiment, the central region 91 includes the second hole 5410, and the central regions of the atomizing room 520 and the first hole 32. The marginal region 92 includes the plurality of first airflow holes 5431, and the peripheral regions of the atomizing room 520 and the first hole 32.

Because the one part of the air flows through the central region 91 in the swirling state and forms the swirling flow 93 in the central region 91, and the other part of the air flows through marginal region 92 in the straight state and forms the straight flow 94 in the marginal region 92, the straight flow 94 keeps stability of the smoke's taste, and the swirling flow 93 in the central region 91 presses the straight flow 94 in the marginal region 92 to facilitate to increase airspeed of the straight flow 94 and improve heat dissipation performance of the atomizer 20. That is to say, the atomizer 20 of the present disclosure keeps the stability of the smoke's taste by making use of the straight flow 94, and increases the airspeed of the straight flow 94 and improves the heat dissipation performance of the atomizer 20 by making use of the swirling flow 93.

Further, the swirling flow 93 scours condensation E-liquid in the accommodating room 420 to a plurality of fine drops, and the fine drops and the swirling flow 93 mix into E-liquid-air mixture flowing through the atomizing member 52 to atomize again, thereby keeping dry and clean of the accommodating room 420. Thai is to say, by taking use of the swirling flow 93 scouring condensation E-liquid in the accommodating room 420 to the fine drops, and the E-liquid-air mixture is atomized by the atomizing member 52 again, thereby keeping dry and clean of the accommodating room 420. Simultaneously, the fine drops is swung to inner walls of atomizing room 520 and the first hole 32 under the function of centrifugal force of the swirling flow 93, and the fine drops and the swirling flow 93 are separated, thereby keeping the cleanliness of the smoke and improving the user's taste experience.

In one embodiment, the atomizing member 52 includes a heating wire 522. The fine drops separated by swirling flow 93 in the central region 91 is scoured to the heating wire 522 to atomize again.

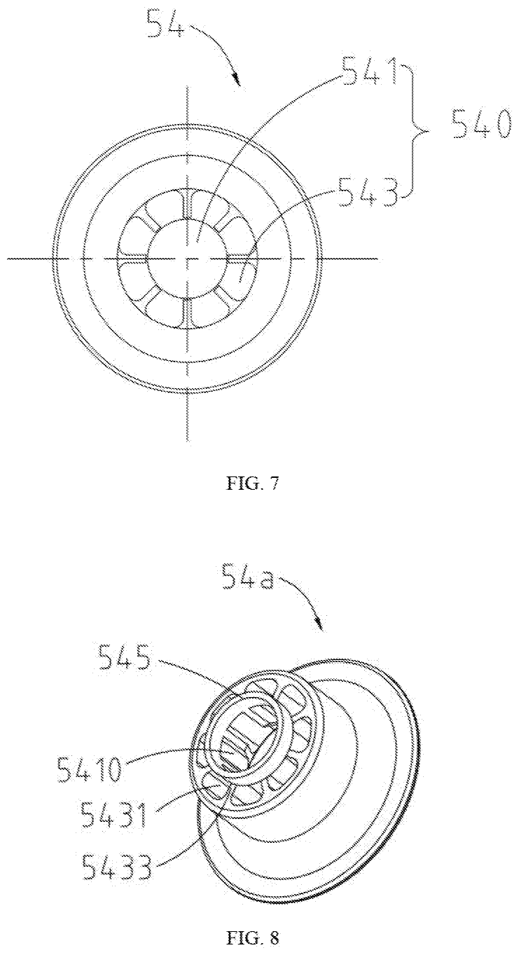

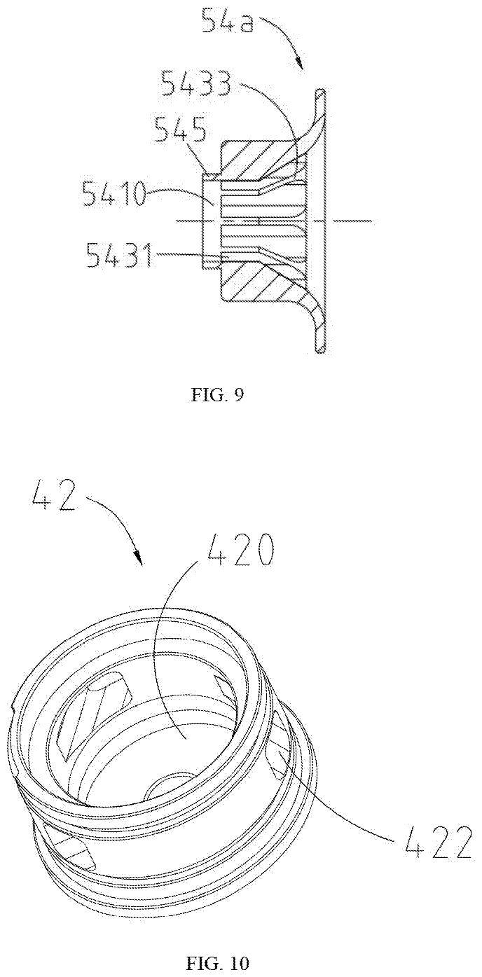

Referring to FIG. 6 to FIG. 9, the FIG. 6 and FIG. 7 disclosure the diverting member 54 in accordance with a first exemplary embodiment of the present disclosure, and the FIG. 8 and FIG. 9 disclosure a diverting member 54a in accordance with a second exemplary embodiment of the present disclosure.

Each of the first diverting plates 5433 is arranged on an inner wall of the diverting room 540 along a radial direction and projects from the inner wall of the diverting room 540 to the second hole 5410, and each of the first diverting plates 5433 is located between two adjacent first airflow holes 5431, to facilitate the other part of the air flows through the marginal region 92 in the straight state.

Further, cross-sections of the plurality of first diverting plates 5433 form a circle, a central axis of the circle is the same as that of the second hole 5410.

In one embodiment, the first diverting plates 5433 are evenly arranged on the inner wall of the diverting room 540 along the radial direction, so that the first airflow holes 5431 are also arranged on the inner wall of the diverting room 540 along the radial direction evenly.

Because the first airflow holes 5431 are evenly arranged on the inner wall of the diverting room 540, the straight flow 94 evenly flows through the marginal region 92 to a mouth of the user, further keeping the stability of the smoke's taste.

Further, a cross-section of each of the plurality of first airflow holes 5431 is a general U shape having a slowly decreasing opening, a central axis of each of the plurality of first airflow holes 5431 is parallel to that of the second hole 5410.

Because, the cross-section of each of the plurality of first airflow holes 5431 is the U shape having the slowly decreasing opening, that is, the cross-section of each of the plurality of first airflow holes 5431 slowly reduces from the inner wall of the diverting room 540 to the second 5410, so that the straight flow 94 in the first airflow holes 5431 does not flow into the second hole 5410, thereby there is a clear boundary between the swirling flow 93 and the straight flow 94.

Further, the diverting member 54a further includes an eliminating noise portion 545 located at a distal end thereof and a border between the swirling flow region 541 and the straight flow region 543.

In one embodiment, the eliminating noise portion 545 can weaken the resonance noise of a air passage. The central region 91 and the marginal region 92 are designed as the air passage.

In one embodiment, the eliminating noise portion 545 projects from a front face of the first diverting plates 5433 to the atomizing room 520.

Referring to FIG. 10 to FIG. 13, the base body 42 includes a swirling flow forming member 424 to change the air from the plurality of inlet holes 422 to the swirling flow 93 to flow through the central region 91.

The swirling flow forming member 424 can change the air from the plurality of inlet holes 422 to the swirling flow 93 in the accommodating room 420, and the swirling flow 93 can scours condensation E-liquid in the accommodating room 420 to the fine drops, and the fine drops and the swirling flow 93 mix into E-liquid-air mixture to enter the atomization core assembly 50, simultaneously, the fine drops is separated to enter the heating wire 522 to atomize again under the function of centrifugal force of the swirling flow 93, thereby keeping the cleanliness of the smoke and improving the user's taste experience.

Further, the swirling flow 93 in the accommodating room 420 is changed to the straight flow 94 after flowing through the first diverting plates 5433, thereby forming the stable straight flow 94 in the straight flow region 543.

In one embodiment, each of the plurality of first diverting plates 5433 is provided to change the swirling flow 93 formed by the swirling flow forming member 424 to the straight flow 94 to flow through the marginal region 92. A cross-section of each of the plurality of first diverting plates slowly expands from the base body 42 to the atomizing member 52 to facilitate the swirling flow 93 to slowly change to the straight flow 94, thereby reducing airflow noise during the swirling flow 93 changing to the straight flow 94.

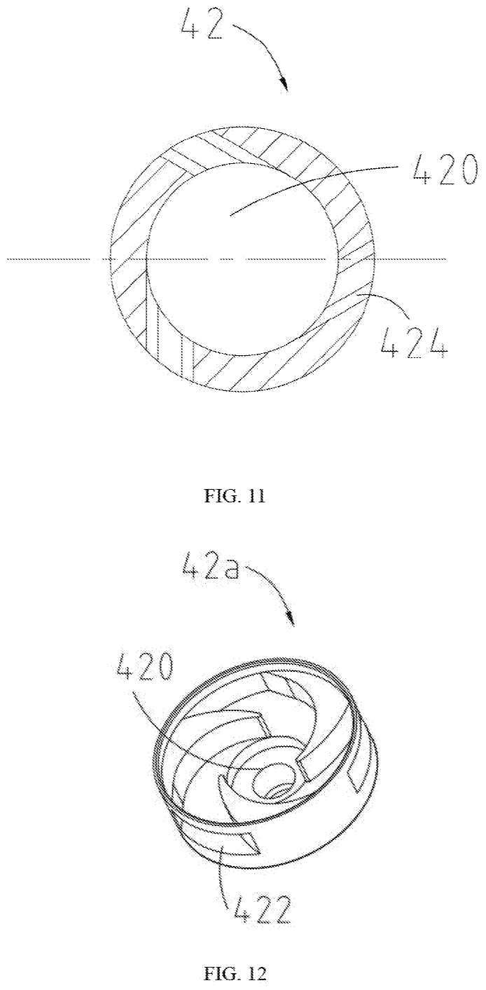

FIG. 10 and FIG. 11 disclose the swirling flow forming member 424 in accordance with a first exemplary embodiment of the present disclosure. The swirling flow forming member 424 is inner walls of the inlet holes 422, wherein each of the inner walls of the inlet holes 422 and the inner wall of the accommodating room 420 are tangent, or direction of the air from the inlet holes 422 getting into the accommodating room 420 is parallel to the tangent line of the inner wall of the accommodating room 420. That is to say, the air from inlet holes 422 enters the accommodating room 420 along the swirling flow forming member 424, and is changed to the swirling flow 93.

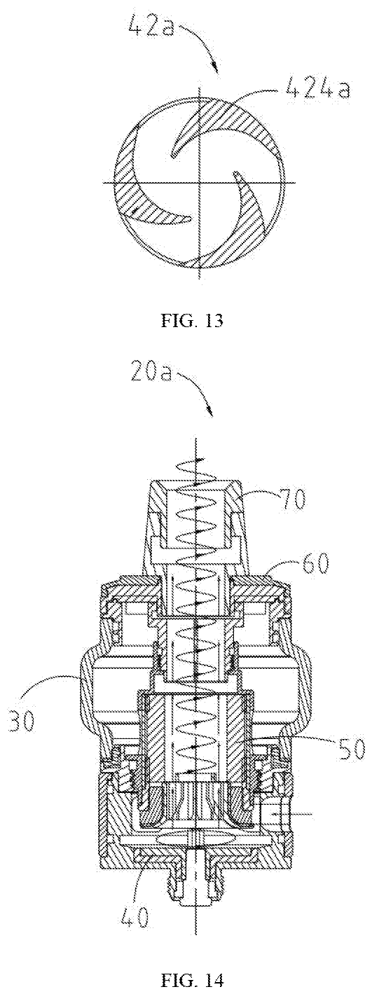

FIG. 12 and FIG. 13 disclose a swirling flow forming member 424a in accordance with a second exemplary embodiment of the present disclosure. The swirling flow forming member 424a includes a plurality of arcuate guide vanes arranged on an inner wall of the accommodating room 420 and extending meanderingly from the inner wall of the accommodating room 420 to a center of the accommodating room 420. That is to say, the air from the inlet holes 422 enters the accommodating room 420 along the swirling flow forming member 424a, and is changed to the swirling flow 93.

In one embodiment, the guide vanes of the swirling flow forming member 424a are similar to guide vanes of a fan or a compressor to change the air from the inlet holes 422 to the swirling flow 93.

In one embodiment, the number of the guide vanes of the swirling flow forming member 424a is three, and the guide vanes are evenly arranged on the inner wall of the accommodating room 420 and extend meanderingly from the inner wall of the accommodating room 420 to the center of the accommodating room 420.

Tops of cross-sections of the guide vanes of the swirling flow forming member 424a form a circle, a central axis of the circle is the same as that of the accommodating room 420.

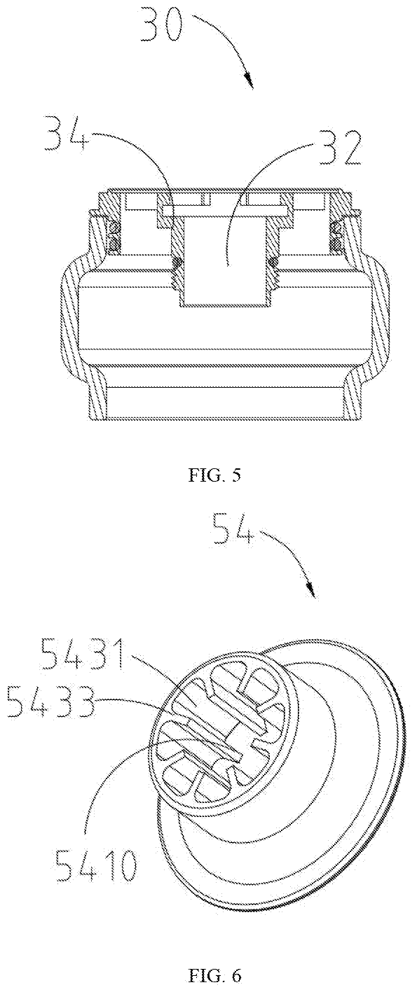

Referring to FIG. 5, the liquid storage tank assembly 30 includes a cover 34, and the first hole 32 is located in a middle portion of the cover 34.

Referring to FIG. 2, FIG. 4, and FIG. 5, the atomizer 20 further includes a deposition chamber 80 located between the cover 34 and the atomization core assembly 50. In detail, the deposition chamber 80 is surrounded by a bottom portion 340 of the cover 34 and a top portion 560 of a housing 56 of the atomization core assembly 50.

Carbon deposition in the heating wire 522 is scoured to a plurality of small carbon particles by the swirling flow 93, and the carbon particles and the swirling flow 93 mix into carbon particle-air mixture to flow through the central region 91. Simultaneously, during the carbon particle-air mixture flowing through the central region 91, the carbon particle-air mixture is separated, and the carbon particles deposit to the deposition chamber 80, thereby keeping cleanliness of the smoke when the user smokes and ensuring health of the user.

Further, when E-liquid together with the swirling flow 93 rises, the bottom portion 340 of the cover 34 block the E-liquid into the deposition chamber 80, thereby effectively preventing E-liquid climbing and avoiding E-liquid to the user's mouth.

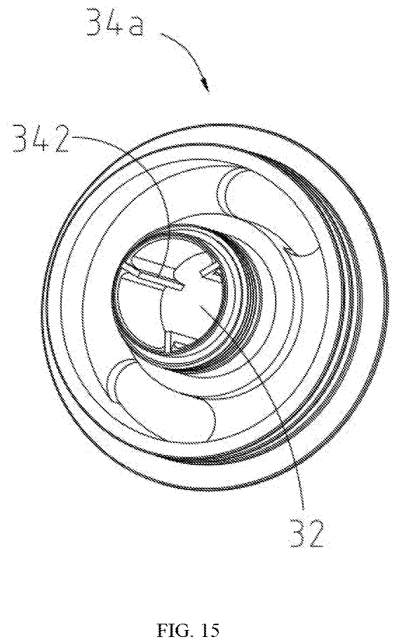

Referring to FIG. 14 and FIG. 15, FIG. 14 discloses a cross-sectional view of an atomizer 20a in accordance with a second exemplary embodiment of the present disclosure, FIG. 15 discloses an isometric view of an cover 34a in accordance with a second exemplary embodiment of the present disclosure. A structure of the atomizer 20a is similar to that of the atomizer 20, and function of the atomizer 20a is the same as that of the atomizer 20, the different between the atomizer 20a and the atomizer 20 is that the atomizer 20a includes the diverting member 54a and the cover 34a. The cover 34a includes a plurality of stabilizing flow plates 342, and the stabilizing flow plates 342 are arranged on an inner wall of the first hole 32 and projecting from the inner wall of the first hole 32 to a center of the first hole 32.

During use, because the straight flow region 543 is adjacent to the base body 42, intensity of straight flow 94 is weaken after flowing a distance, the stabilizing flow plates 342 of the cover 34a is provided to stabilize the intensity of straight flow 94.

In other embodiment, if the distance between the diverting member 54 and the cover 34a is shorter, the stabilizing flow plates 342 is not need.

Referring to FIG. 2, the atomizer 20 further includes a top cover assembly 60 and a cigarette holder 70. The top cover assembly 60 is assembled between the cigarette holder 70 and liquid storage tank assembly 30.

Referring to FIG. 1 to FIG. 15, in assembly, the diverting member 54 is assembled in the atomizing member 52 to mount to the atomization core assembly 50. The atomization core assembly 50 is mounted to the base body 42. The liquid storage tank assembly 30 is mounted to the base assembly 40, and the atomization core assembly 50 is received between the liquid storage tank assembly 30 and the base assembly 40. The top cover assembly 60 is assembled between the cigarette holder 70 and liquid storage tank assembly 30. In this way, the liquid storage tank assembly 30, the base assembly 40, the atomization core assembly 50, the top cover assembly 60, and the cigarette holder 70 are assembled to the atomizer 20. The atomizer 20 is then connected to the battery assembly 10, thereby obtaining the aspiration device with atomization function 100.

In use, after the air from the inlet holes 422 flows through the swirling flow forming member 424 of the base assembly 40, the swirling flow 93 is formed. After the swirling flow 93 flows through the diverting member 54, one part of the air flows through the central region 91 to the cigarette holder 70 in the swirling state, and the other part of the air flows through marginal region 92 to the cigarette holder 70 in the straight state.

The aspiration device with atomization function 100 can form the straight flow 94 and the swirling flow 93, thereby keeping stability of the smoke's taste and improving heat dissipation performance to prolong the life of the aspiration device with atomization function 100, and aspiration device with atomization function 100 have performance of dry and clean, preventing E-liquid leakage and pollution, and improving comfort of use. Further, eliminating noise portion 545 can weaken the resonance noise of the air passage, thereby enhancing experience and auditory comfort of the user.

Although the features and elements of the present disclosure are described as embodiments in particular combinations, each feature or element can be used alone or in other various combinations within the principles of the present disclosure to the full extent indicated by the broad general meaning of the terms in which the appended claims are expressed.

What is claimed is:

1. An atomizer, comprising:
a central region;
a marginal region;
a liquid storage tank assembly defining a first hole;
a base assembly comprising a base body, the base body comprising an accommodating room and a plurality of inlet holes in communication with the accommodating room; and
an atomization core assembly located between the liquid storage tank assembly and the base assembly, the atomization core assembly comprising an atomizing member and a diverting member assembled in the atomizing member, the atomizing member comprising an atomizing room, the diverting member comprising a diverting room comprising a swirling flow region and a straight flow region in communication with the swirling flow region, the swirling flow region defining a second hole, the straight flow region comprising a plurality of first airflow holes and a plurality of first diverting plates, the second hole and the plurality of first airflow holes in communication with the atomizing room;
wherein air flows into the accommodating room from the plurality of inlet holes, one part of the air flows through the second hole, the atomizing room, and the first hole in a swirling state, and forms a swirling flow in the central region; the other part of the air flows through the plurality of first airflow holes, the atomizing room, the first hole in a straight state, and forms a straight flow in the marginal region.

2. The atomizer as claimed in claim 1, wherein each of the first diverting plates is arranged on an inner wall of the diverting room along a radial direction and projects from the inner wall of the diverting room to the second hole, and each of the first diverting plates is located between two adjacent first airflow holes, to facilitate the other part of the air flows through the marginal region in the straight state.

3. The atomizer as claimed in claim 2, wherein cross-sections of the plurality of first diverting plates form a circle, a central axis of the circle is the same as that of the second hole.

4. The atomizer as claimed in claim 2, wherein a cross-section of each of the plurality of first airflow holes is a general U shape having a slowly decreasing opening, a central axis of each of the plurality of first airflow holes is parallel to that of the second hole.

5. The atomizer as claimed in claim 1, wherein the diverting member further comprises an eliminating noise portion located at a distal end thereof and a border between the swirling flow region and the straight flow region.

6. The atomizer as claimed in claim 1, wherein the base body comprises a swirling flow forming member to change the air from the plurality of inlet holes to the swirling flow to flow through the central region in the swirling state.

7. The atomizer as claimed in claim 6, wherein each of the plurality of first diverting plates is provided to change the swirling flow formed by the swirling flow forming member to the straight flow to flow through the marginal region in the straight state, and a cross-section of each of the plurality of first diverting plates slowly expands from the base body to the atomizing member to facilitate the swirling flow to slowly change to the straight flow.

8. The atomizer as claimed in claim 6, wherein the swirling flow forming member comprises a plurality of arcuate guide vanes arranged on the inner wall of the accommodating room and extending meanderingly from the inner wall of the accommodating room to a center of the accommodating room.

9. The atomizer as claimed in claim 1, wherein the liquid storage tank assembly comprises a cover, and the first hole is located in a middle portion of the cover, and wherein the cover comprises a stabilizing flow plate, and the stabilizing flow plate is arranged on an inner wall of the first hole and projecting from the inner wall of the first hole to a center of the first hole.

10. The atomizer as claimed in claim 9, wherein the atomizer further comprises a deposition chamber located between the cover and the atomization core assembly to receive a plurality of small carbon particles.

11. An aspiration device with atomization function, comprising:
a battery assembly; and
an atomizer connected to the battery assembly, the atomizer comprising:
a central region;
a marginal region;
a liquid storage tank assembly defining a first hole;
a base assembly comprising a base body, the base body comprising an accommodating room and a plurality of inlet holes in communication with the accommodating room; and
an atomization core assembly located between the liquid storage tank assembly and the base assembly, the atomization core assembly comprising an atomizing member and a diverting member assembled in the atomizing member, the atomizing member comprising an atomizing room, the diverting member comprising a diverting room comprising a swirling flow region and a straight flow region in communication with the swirling flow region, the swirling flow region defining a second hole, the straight flow region comprising a plurality of first airflow holes and a plurality of first diverting plates, the second hole and the plurality of first airflow holes in communication with the atomizing room;
wherein air flows into the accommodating room from the plurality of inlet holes, one part of the air flows through the second hole, the atomizing room, and the first hole in a swirling state, and forms a swirling flow in the central region; the other part of the air flows through the plurality of first airflow holes, the atomizing room, the first hole in a straight state, and forms a straight flow in the marginal region.

12. The aspiration device with atomization function as claimed in claim 11, wherein each of the first diverting plates is arranged on an inner wall of the diverting room along a radial direction and projects from the inner wall of the diverting room to the second hole, and each of the first diverting plates is located between two adjacent first airflow holes, to facilitate the other part of the air flows through the marginal region in the straight state.

13. The aspiration device with atomization function as claimed in claim 12, wherein cross-sections of the plurality of first diverting plates form a circle, a central axis of the circle is the same as that of the second hole.

14. The atomizer as claimed in claim 12, wherein a cross-section of each of the plurality of first airflow holes is a general U shape having a slowly decreasing opening, a central axis of each of the plurality of first airflow holes is parallel to that of the second hole.

15. The aspiration device with atomization function as claimed in claim 11, wherein the diverting member further comprises an eliminating noise portion located at a distal end thereof and a border between the swirling flow region and the straight flow region.

16. The aspiration device with atomization function as claimed in claim 11, wherein the base body comprises a swirling flow forming member to change the air from the plurality of inlet holes into the swirling flow to flow through the central region in the swirling state.

17. The aspiration device with atomization function as claimed in claim 16, wherein each of the plurality of first diverting plates is provided to change the swirling flow formed by the swirling flow forming member to the straight flow to flow through the marginal region in the straight state, and a cross-section of each of the plurality of first diverting plates slowly expands from the base body to the atomizing member to facilitate the swirling flow to slowly change to the straight flow.

18. The aspiration device with atomization function as claimed in claim 16, wherein the swirling flow forming member comprises a plurality of arcuate guide vanes arranged on the inner wall of the accommodating room and extending meanderingly from the inner wall of the accommodating room to a center of the accommodating room.

19. The aspiration device with atomization function as claimed in claim 11, wherein the liquid storage tank assembly comprises a cover, and the first hole is located in a middle portion of the cover, and wherein the cover comprises a stabilizing flow plate, and the stabilizing flow plate is arranged on an inner wall of the first hole and projecting from the inner wall of the first hole to a center of the first hole.

20. The aspiration device with atomization function as claimed in claim 19, wherein the atomizer further comprises a deposition chamber located between the cover and the atomization core assembly to receive a plurality of small carbon particles.
